(19) United States
(12) Patent Application Publication
(10) Pub. No.: US 2006/0018843 A1
(43) Pub. Date: Jan. 26, 2006

(54) Halitosis, gingivitis, and periodontitis treatment and preventative composition

(76) Inventor: Kenneth D. Fine, Dallas, TX (US)

Correspondence Address: STORM L.L.P., BANK OF AMERICA PLAZA, 901 MAIN STREET, SUITE 7100, DALLAS, TX, 75202, US

(21) Appl. No.: 11/187,275

(22) Filed: Jul. 22, 2005

Related U.S. Application Data
(60) Provisional application No. 60/590,617, filed on Jul. 24, 2004.

(51) Int. Cl. A61K 8/96; A61K 35/74

(52) U.S. Cl. 424/48; 424/50; 424/93.45

(57) ABSTRACT

Disclosed is a composition and method for the treatment and prevention of bad breath, halitosis, gingivitis, and/or periodontitis that contains one or more varieties of probiotic bacteria from the groups Lactobacillus, Bacillus, Escherichia, Enterococcus, Streptococcus, and Bifidobacterium, and/or a species of the yeast genus Saccharomyces. The composition may be in a variety of forms, such as a toothpaste, mouthwash, oral spray, oral cream or gel, chewing gum, candy, lozenges, dissolvable pill or strip, or powder that may be sprinkled directly into the oral cavity.

### CLAIM OF PRIORITY

This application claims priority from U.S. Provisional Patent Application No. 60/590,617 entitled “HALITOSIS, GINGIVITIS, AND PERIODONTITIS TREATMENT AND PREVENTATIVE COMPOSITION” filed on behalf of Dr. Kenneth Fine on Jul. 24, 2004 (Attorney Docket No. FINE 02984 PTPV).

### FIELD OF THE INVENTION

The present invention relates generally to the field of oral hygiene. More specifically, this invention relates to a composition which includes as its active ingredient a variety of one or more probiotic organisms from the groups Lactobacillis, Bacillus, Escherichia, Enterococcus, Streptococcus, Saccharomyces, and Bifidobacterium, for use in the prevention and elimination of bad breath (halitosis), gingivitis, and periodontitis.

### BACKGROUND

There are many varieties of bacteria that inhabit the body of humans. Some are “good” bacteria that help the body perform various functions, such as aiding in digestion of food in the gastrointestinal system. Others are “bad” bacteria that cause infections, diseases and other disorders, especially in the gastrointestinal tract and respiratory system.

In the oral cavity, “bad” bacteria are responsible for, among other things, halitosis, gingivitis, and periodontitis. Bad breath, which affects tens of millions of people, can be attributed to a variety of causes, including eating odiferous foods, poor oral hygiene, throat infections and tooth decay. Halitosis is a condition of chronic bad breath. While more frequent flossing and brushing of the teeth, gums, cheeks, and tongue can help reduce the problem by eliminating food particles that can be a cause of bad breath, this does not solve the problem in all cases. In many cases, bad breath can be traced to bacteria in the mouth, and the toxins they produce.

Gingivitis is inflammation of the gums which, if left untreated, can lead to periodontitis, in which the inflammation spreads from the gums to the ligaments and bones in the mouth. Gingivitis and periodontitis are caused by plaque deposits. Plaque is a sticky material that develops on the exposed portions of the teeth, consisting of bacteria, mucus, and food debris. Bacteria and the toxins they produce cause the gums to become infected, swollen, and tender.

Among other things the bad bacteria generate as waste products hydrogen sulfide, methyl mercapatan and other sulfur gases. These gases are believed to be one of the major contributing causes of the odor of halitosis;and directly related to the importance and mechanism of this invention, these sulfur gases are the same class of gases generated in the distal small intestine and colon that give feces their bad odor. The gases may also be the cause of tissue damage in the mouth that occurs in gingivitis, and colitis in the colon. It is known to use chemical compounds such as chlorine dioxide or zinc in the oral cavity to reduce the concentration of sulfur compounds in the mouth, and thus reduce or eliminate the odor. For example, U.S. Pat. No. 5,833,952 to Grigor et al. discloses use of a stannous compound in conjunction with a zinc compound to combat bad breath. Similarly, U.S. Pat. No. 6,231,836 to Takhtalian et al. discloses a dentrifice for controlling gum disease and bad breath that includes, among other ingredients, zinc.

Many tools and chemicals have been developed for the treatment of these various disorders. However, many are not effective, and others are very expensive or complicated.

Accordingly, a continuing search has been directed to the development of methods and treatments which can reduce or eliminate halitosis, gingivitis, and periodontitis and that are simple and cost-effective.

### SUMMARY OF THE INVENTION

One object of this invention is to provide a method that is effective in the treatment of bad breath in mammals. It is a further object of the present invention to provide a method and treatment that is effective in the treatment of gingivitis and periodontitis in humans or other mammals. It is yet a further object of the present invention to provide a treatment and method that is simple and inexpensive. It is yet a further object of the present invention to provide a treatment and method that utilizes products that are effective and safe to the environment and the user. It is also an object of the present invention that it is palatable to the user.

The present invention provides for a method or a composition for the treatment or prophylaxis of halitosis, gingivitis and periodontitis in the oral cavity of a mammal comprising the application of a compound comprising one or more probiotic bacteria to the oral cavity. The mechanism will involve local action in the mouth but also by its similar action in the distal small intestine and colon.

In another preferred embodiment of the present invention, the probiotic organisms are selected from a species of the genera consisting of Lactobacillus, Bacillus, Escherichia, Enterococcus, Streptococcus, and Bifidobacterium bacteria, and/or a species of the yeast genus Saccharomyces.

In yet another preferred embodiment of the present invention, the mammal is selected from the group consisting of human, cat, and dog.

In some other preferred embodiments of the present invention, the compound is a mouth wash, toothpaste, chewing gum, lozenge, or powder.

In another preferred embodiment of the present invention, a treatment for halitosis, gingivitis and periodontitis in an oral cavity of a mammal is provided. The treatment comprises applying prokaryotes for inhibiting the growth of pathogenic bacteria in the oral cavity, as well as establishing the favorable growth of the selected organism.

In another preferred embodiment of the present invention, the prokaryotes further comprises pH increasing prokaryotes.

In yet another preferred embodiment of the present invention, the means for biochemically treating further comprises bacteria, where, in another preferred embodiment, the bacteria further comprises a species of Lactobacillus, Bacillus, Escherichia, Enterococcus, Streptococcus, and/or Bifidobacterium, and/or a species of the yeast genus Saccharomyces.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION

The present invention, administering probiotic “good” bacteria in the oral cavity inhibits growth or enables reduction of less favorable “bad” bacteria in the oral cavity, which consequently reduces or prevents halitosis, gingivitis and/or periodontitis. By doing this, the present invention may help to break down, or at least, inhibit or minimize “bad” bacterial waste products such as hydrogen sulfide, methyl mercapatan and other sulfur gases. These gases are believed to be one of the major contributing causes of the odor of halitosis. The gases may also be the cause of tissue damage in the mouth that occurs in gingivitis. Administering probiotics in the mouth also has the same probiotic effect on the rest of the gastrointestinal tract because the orally applied products will be swallowed. It is stated in this invention that we have discovered that halitosis, in part, is caused by the generation of these odiferous sulfur gases and other odiferous gases by fecal organisms (very similar to oral cavity organisms) in the distal small intestine and colon, which then get absorbed across the intestinal mucosa, circulate in the blood, and are excreted by the lungs in exhaled air (breath). Therefore, the administered probiotics in the invention will improve breath odor not only by the above mentioned mechanisms in the mouth itself, but also by a similar action in the distal gastrointestinal tract as well.

The use of “good” bacteria for the treatment of infections is known. Many antibiotics are derived from various species of bacteria. Additionally, ingestion of certain “good” bacteria that are typically found in the body, sometimes referred to as “probiotics,” for treatment or prevention of gastrointestinal or other disorders is known. For example, ingestion of Lactobacillus acidophilus or other probiotic bacteria is recommended to improve the digestion of milk products, especially in persons who are lactose-intolerant, because it helps break down milk sugar. Ingestion of Lactobacillus acidophilus is often recommended after treatment with oral antibiotics to help replenish the level of “good” bacteria in the digestive system that were killed by the antibiotics, and to reduce or eliminate the side effects experienced by persons who have taken antibiotics, such as diarrhea. In addition to the use of bacteria, it is also possible to utilize various prokaryotes.

However, although the bad bacteria are a cause of many incidents of halitosis, and are also a factor in gingivitis and periodontitis, the introduction of “good” bacteria into the oral cavity to eliminate or inhibit the growth of the “bad” bacteria that cause halitosis, gingivitis or periodontitis has not been previously considered.

The composition of the present invention, which includes a variety of one or more probiotic bacteria from the groups Lactobacillus, Bacillus, Escherichia, Enterococcus, Streptococcus, and Bifidobacterium, and/or a species of the yeast genus Saccharomyces in an orally acceptable medium, possibly with other ingredients, can take the form of a mouthwash, oral rinse, oral spray, toothpaste, oral cream or gel, chewing gum, candy, lozenges, dissolvable pill or strip, or powder that may be sprinkled directly into the oral cavity or added to food. The invention will assist in inhibiting the growth of the bad bacteria that cause halitosis and gingivitis. It is believed that the invention works by doing one or more of the following: 1) encouraging the growth of probiotic bacteria, which in turn inhibits the growth of more harmful bacteria, such as Bacteroides and other genera in the oral cavity and the distal gastrointestinal tract; 2) raising the pH of the saliva in the mouth, which discourages growth of the bacteria; 3) increasing production of saliva, which helps to wash away bacteria that may grow in the oral cavity, and the toxins generated by those bacteria; and 4) breaking down the toxins generated by the bacteria.

Optionally, other ingredients may be used, such as flavoring agents, coloring agents, anti-caries (cavity) agents, anti-staining compounds, natural compounds such as folic acid, which are known to bind to the toxins secreted by plaque bacteria, thymol and/or eucalyptol which are known to kill bacteria and break down sulfur compounds that are generated by the bacteria, and inert ingredients such as water, thickening agents, gums, carriers or binders, depending on the form used for the invention.

In use, the compound is inserted into the oral cavity on a regular basis. For example, a toothpaste embodiment of the present invention would be used to brush the teeth with a toothbrush, typically at least twice per day. A mouthwash embodiment of the present invention would be poured into the oral cavity and swished in the mouth for 30-60 seconds. An oral spray, cream or gel embodiment would be sprayed or squirted directly into the mouth. A chewing gum embodiment would be chewed, releasing the ingredients into the mouth. A candy or lozenge embodiment would be inserted into the mouth and sucked on to dissolve the product and deliver the invention to the oral cavity. A dissolvable pill or strip embodiment of the invention would be placed in the mouth and held there until the carrier dissolved, thereby releasing the invention into the oral cavity. A powder embodiment of the present invention could be sprinkled directly into the oral cavity, or added to food.

Thus, the present invention allows for a more healthy solution to oral hygiene. By introducing probiotic bacteria or other such prokaryotes, such as Lactobacillus, Bacillus, Escherichia, Enterococcus, Streptococcus, Saccharomyces, and Bifidobacterium the natural flora of the person or animal can be more healthfully developed and maintained. This in turn provides a more stable and beneficial treatment compared to standard chemical treatments.

It is understood that the present invention can take many forms and embodiments. Accordingly, several variations may be made in the foregoing without departing from the spirit or the scope of the invention. Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments. Accordingly, it is appropriate that the appended claims be construed broadly and in a manner consistent with the scope of the invention.

1. A method for the treatment or prophylaxis of halitosis, gingivitis and periodontitis in an oral cavity of a mammal comprising application of a compound comprising one or more probiotic organisms to said oral cavity.

2. The method of claim 1, wherein said probiotic organisms are selected from a species of the group consisting of Lactobacillus, Bacillus, Escherichia, Enterococcus, Streptococcus, and Bifidobacterium, and Saccharomyces.

3. The method of claim 1, wherein said mammal is selected from the group consisting of human, cat, and dog.

4. The method of claim 1, wherein said compound is a mouthwash.

5. The method of claim 1, wherein said compound is a toothpaste.

6. The method of claim 1, wherein said compound is a chewing gum.

7. The method of claim 1, wherein said compound is a lozenge.

8. The method of claim 1, wherein said compound is a powder.

9. A composition for treatment or prophylaxis of halitosis, gingivitis and periodontitis in an oral cavity of a mammal comprising one or more probiotic organisms to said oral cavity.

10. The composition of claim 9, wherein said probiotic organisms are selected from a species of the group consisting of Lactobacillus, Bacillus, Escherichia, Enterococcus, Streptococcus, and Bifidobacterium, and Saccharomyces.

11. The composition of claim 9 wherein said compound is a mouth wash.

12. The composition of claim 9 wherein said compound is a toothpaste.

13. The composition of claim 9 wherein said compound is a chewing gum.

14. The composition of claim 9 wherein said compound is a lozenge.

15. The composition of claim 9 wherein said compound is a powder.

16. A treatment for halitosis, gingivitis and periodontitis in an oral cavity of a mammal comprising applying prokaryotes for inhibiting growth of pathogenic bacteria in said oral cavity.

17. The treatment of claim 16, wherein said prokaryotes further comprises pH increasing prokaryotes.

18. The treatment of claim 16, wherein said prokaryotes further comprises bacteria.

19. The treatment of claim 18, wherein said bacteria further comprises a species of Lactobacillus, Bacillus, Escherichia, Enterococcus, Streptococcus, and Bifidobacterium.

20. The treatment of claim 16, wherein said prokaryotes further comprises yeast.

21. The treatment of claim 18, wherein said yeast further comprises a species Saccharomyces.
